(19) United States
(12) Patent Application Publication
(10) Pub. No.: US 2024/0302379 A1
(43) Pub. Date: Sep. 12, 2024

(54) METHODS FOR IDENTIFYING AND MONITORING INTERACTIONS OF PROTEIN WITH LIGAND

(71) Applicant: Agency for Science, Technology and Research, Singapore (SG)

(72) Inventor: Soon Heng Tan, Singapore (SG)

(21) Appl. No.: 18/258,775

(22) PCT Filed: Dec. 20, 2021
(86) PCT No.: PCT/SG2021/050807

§ 371 Date: Jun. 21, 2023

(30) Foreign Application Priority Data
Dec. 24, 2020 (SG) 10202013042R

(51) Int. Cl. G01N 33/68

(52) U.S. Cl. CPC G01N 33/6803; G01N 2800/52

(57) ABSTRACT

The invention relates generally to the field of biochemistry. In particular, the invention relates to a method of detecting or measuring target that is bound to a ligand in a sample, the method comprising contacting a sample comprising one or more cells with a cell-permeable denaturant to promote intracellular unfolding of the target in the sample, followed by lysing the sample. The lysed sample is then detected or measured for the level of non-aggregated target or aggregated target, wherein a difference in the level of non-aggregated target or aggregated target as compared to a reference indicates the presence or level of target that is bound to the ligand in the sample. In specific embodiments, the cell-permeable denaturant is urea or derivatives thereof. Methods of identifying a candidate ligand or predicting the efficacy of a drug in a subject are also provided therein.

### FIELD OF INVENTION

The invention relates generally to the field of biochemistry. In particular, the invention relates to a method of detecting or measuring target that is bound to a ligand in a sample. Methods of identifying a candidate ligand or predicting the efficacy of a drug in a subject are also provided herein.

### BACKGROUND

The ability to identify and monitor protein-chemical interactions has many important applications in biology, chemistry and drug discovery. For example, it is important to monitor protein-chemical interactions when screening large chemical libraries for drug discovery and subsequent development. It is also important to understand off-target interactions of drugs with other proteins that may result in undesired side effects.

Existing approaches to identify and monitor protein-chemical interactions, however, often require the use of recombinant proteins and modified bioactive compounds. This may compromise the physiological relevance of the data obtained. For example, drugs that are screened and identified with recombinant proteins may not engage their targets intracellularly. Drugs identified with recombinant proteins may have unknown off-target activities that lead to toxicity. Recombinant proteins may also adopt different structural conformations outside the cell and may be challenging to express. Furthermore, the mechanism of action or protein targets of bioactive compounds identified from phenotypic drug screens are often unknown.

Accordingly, it is generally desirable to overcome or ameliorate one or more of the above mentioned difficulties.

### SUMMARY

Disclosed herein is a method of detecting or measuring a target that is bound to a ligand in a sample, the method comprising:
a) contacting a sample with a cell-permeable denaturant to promote intracellular unfolding of the target in the sample,
b) lysing the sample; and
c) detecting or measuring the level of non-aggregated target or aggregated target, wherein a difference in the level of non-aggregated target or aggregated target as compared to a reference indicates the presence or level of target that is bound to the ligand in the sample.

Disclosed herein is a kit for performing a method as defined herein.

Disclosed herein is a method of identifying a candidate ligand that is capable of binding to a target, the method comprising:
a) contacting a sample with the candidate ligand;
b) contacting the sample with a cell-permeable denaturant to promote intracellular unfolding of the target;
c) lysing the sample; and
d) detecting or measuring the level of the non-aggregated target or aggregated target, wherein a difference in the level of non-aggregated target or aggregated target as compared to a reference indicates that the candidate ligand is capable of binding to the target.

Disclosed herein is a method of predicting the efficacy of a drug in a subject, the method comprising
a) obtaining a sample from a subject who has been treated with the drug;
b) contacting the sample with a cell-permeable denaturant to promote intracellular unfolding of a target;
c) lysing the sample;
d) detecting or measuring the level of non-aggregated target or aggregated target, wherein a difference in the level of non-aggregated target or aggregated target as compared to a reference indicates binding of the drug to the target, therefore predicting efficacy of the drug in the subject.

Disclosed herein is a method of identifying a target that is bound to a drug in a subject, the method comprising:
a) obtaining a sample from a subject who has been treated with the drug;
b) contacting the sample with a cell-permeable denaturant to promote intracellular unfolding of the target;
c) lysing the sample; and
d) detecting or measuring the level of non-aggregated target or aggregated target, wherein a difference in the level of non-aggregated target or aggregated target as compared to a reference indicates binding of the drug to the target.

### BRIEF DESCRIPTION OF DRAWINGS

Embodiments of the present invention are hereafter described, by way of non-limiting example only, with reference to the accompanying drawings in which:

FIG. 1: Overview of problems faced and addressed by Universal Cellular Profiling (UCEP) technology in drug discovery.

FIG. 2: Diagram illustrates the basic workflow of UCEP for identifying drug target protein based on the physical stability of a protein in presence (and absence) of drug.

FIG. 3: A diagram showing the workflow of the UCEP-Screen system. Firstly, reporter cells are generated using either Flp-In T-ReX or CRISPR techniques. Then, reporter cells are optimized for ideal UCEP conditions before the large-scale small-molecule screen.

FIG. 4: Representative blots of UCEP-ENGAGE for Molarity-Response (MR) experiments. All experiments were run in one biological replicate (n=1). Either GAPDH or alpha-tubulin was used as loading control. (A) Abundance of soluble DHFR was significantly higher in MTX treatment than control at condition of 3M, 4M and 5M. It indicated that DHFR was strongly stabilized by MTX in K562 cells. (B) Abundance of soluble TS was significantly higher in MTX treatment than control at condition of 4M and 5M. It indicated that TS was stabilized by MTX in K562 cells. (C) Abundance of soluble HDAC2 was higher in PAN treatment than control at condition of 4M, 5M and 7M. It showed that HDAC2 was stabilized by PAN in HEPG2 cells. (D) Abundance of soluble ABL kinase and BCR-ABL fusion proteins were significantly higher in dasatinib-treated group than DMSO-treated control group at urea concentrations from 3M to 7M. It indicated that both ABL and BCR-ABL proteins are stabilized by dasatinib.

FIG. 5: UCEP dose response experiments for determining target binding affinity of MTX and PAN. (A) Gradual increase of DHFR band intensity in dose-dependent manner from 0 to 40 uM of MTX. (B) Likewise, increasing abundance of soluble HDAC2 from 0 to 10 uM was also observed in panobinostat treated cells where GAPDH was used as a loading control. Band intensities were semi-quantified using Image Lab and the dose-response curve was fitted via Graphpad for EC50 calculation.

FIG. 6: Volcano plots of UCEP-ID for MR experiment. (A) Target deconvolution for methotrexate in K562 (n=1). Dihydrofolate dehydrogenase (DHFR) was detected as only drug binding target of MTX. (B) Target deconvolution for panobinostat in HepG2 (n=1). A number of targets passed the filter criteria and detected to be stabilized by panobinostat. They were HDAC1, HDAC2, TTC38, HDAC6, CAVIN1, PAH, and ADH5. (C) Target deconvolution for panobinostat in HepG2 with addition of NP40 in dilution buffer (n=2). ER membrane proteins FADS1 and FADS2 were identified with increased protein coverage. (D) Target deconvolution for dasatinib in K562 (n=2). Known direct targets, ABL and BTK kinases were detected as binding targets.

FIG. 7: Validation of UCEP assay development. (A) The effect of five different compounds on protein stability was assessed with or without UCEP in HEK293 DHFR-HiBiT cells. Cells were treated with 20 μM of compounds for 10 minutes. (B) Chemical structure of selective DHFR inhibitors Methotrexate and Aminopterin. (C) Chemical structure of non-DHFR inhibitors Staurosporine, Enzalutamide, and Panobinostat. DHFR: Dihydrofolate reductase

FIG. 8: Comparison of different chemical denaturants. HEK293 DHFR-HiBIT cells treated with 20 μM Methotrexate for 10 minutes followed by UCeP. In UCeP, 3M urea, n-methylurea, guanidine hydrochloride, or guanidinium thiocyanate was used. PBS was used as a vehicle. The result shows a fold change in protein stability. DHFR: Dihydrofolate reductase

FIG. 9: Comparison of the efficiency of protein aggregates separation by magnetic microbeads with centrifugation approach. Results showed that magnetic microparticles could preferentially capture DHFR aggregates (unbound protein) at 4M denaturing condition as efficient as centrifugation method. However, alpha-tubulin which is not the drug target was also completely pulled down by SIMAG-C1 while unaffected with SIMAG-S. It indicated that different surface chemistry of magnetic beads may absorb the soluble fractions of proteins onto the beads, particularly beads coated with alkyl group.

FIG. 10: Measurement of drug binding affinities in reporter cells at different effective urea molarity. (A) HDAC1-HiBiT reporter cells were treated with different doses of panobinostat for 5 minutes followed by UCEP at 4M, 5M, 6M, and 7M urea. Binding affinities calculated from using different urea concentrations were similar and within experimental variation (n=3). (B) DHFR-HiBiT reporter cells were treated with different concentration of aminopterin for 10 minutes followed by UCEP with 2M, 3M, 4M, and 5M urea. Aminopterin showed similar binding affinity among different urea concentrations used (n=2). Data represented as mean±SEM.

FIG. 11: Tucatinib treatment increased stability of the protein target in HER2-HiBIT reporter cells in the presence of 1% CHAPS from 3M to 5M urea. Measured bioluminescence signal values from treatment was divided by values from the control group to calculate fold change and plotted in Graphpad.

### DETAILED DESCRIPTION

The present specification teaches a method of detecting or measuring a target that is bound to a ligand in a sample. The method may comprise a) contacting a sample with a cell-permeable denaturant to promote intracellular unfolding of the target in the sample. The method may comprise b) detecting or measuring the level of non-aggregated target or aggregated target, wherein a difference in the level of non-aggregated target or aggregated target as compared to a reference indicates the presence or level of target that is bound to the ligand in the sample. The method may comprise lysing the sample prior to detecting or measuring the level of non-aggregated target.

Disclosed herein is a method of detecting or measuring a target that is bound to a ligand in a sample, the method comprising: a) contacting a sample with a cell-permeable denaturant to promote intracellular unfolding of the target in the sample, b) lysing the sample; and c) detecting or measuring the level of non-aggregated target or aggregated target, wherein a difference in the level of non-aggregated target or aggregated target as compared to a reference indicates the presence or level of target that is bound to the ligand in the sample.

Without being bound by theory, the invention describes series and combination of steps that permit the use of cell-permeable chemical denaturants to identify and monitor chemical-protein interactions in cell lysate and in cells. Cell-permeable chemical denaturants may be used to unfold protein intracellularly in presence of chemical/drug, followed by rapid cell lysis to dilute the denaturants leading to protein precipitations which are then separated from soluble proteins through centrifugation, filtration or microbeads. A chemical binding to a protein may change the physical stability of the protein that affect the aggregation or precipitation propensity of the protein compared to unbound protein which is exploited to identifying interacting proteins. Other chemicals or physical particles like microbeads or similar material can be added during lysis to enhance aggregation and precipitation.

The step of lysing can be done by addition of cell lysis buffer, rapid freeze-thawing of samples and/or mechanical lysis techniques (such as by passing the sample through the syringe). In one embodiment, the step of lysing is a rapid cell lysis technique. The step of lysis may lead to rapid dilution of the cell-permeable denaturant. In one embodiment, the step of lysing the sample induces aggregation of unfolded target. The step of lysis allows the dilution of the chemical denaturant and the extraction of target to be performed in a single step.

The step of lysing is preferably a non-denaturing lysis technique, allowing target proteins to retain a native i.e. correctly folded or native-like conformation. This is referred to herein as native lysis. This can be carried out chemically or otherwise using reagents which are well known in the art e.g. lysozyme and detergents. The degree of lysis must be sufficient to allow proteins of the cell to pass freely out of the cell. Typically, when dealing with membrane bound proteins, lysis is performed in the presence of detergents or amphiphiles, for example Triton X-100 or dodecylmaltoside, to release the protein from the membrane. The lysis step can alternatively be carried out by freeze thawing the cells. More preferably, lysis is carried out using both native lysis buffer and freeze thawing the cells. Preferably, the lysis buffer contains lysozyme, for examples at 50-750 μg/ml, more preferably at 100-200 μg/ml. DNAse can also be found in native lysis buffer preferably at 250-750 μg/ml. Native lysis buffer may contain for example 20 mM Tris, pH 8, 100 mM NaCl, lysozyme (200 μg/ml) and DNAse I (750 μg/ml). For target proteins known to be inserted into cellular membranes, detergents would be added to the lysis buffer at typical concentrations where they are known to solubilise membrane-inserted proteins in a native form, such as 1% n-dodecyl-β-maltoside. The step of freeze thawing is preferably repeated, i.e. two or more cycles, preferably 3 or more cycles of freeze thawing are performed.

In one embodiment, the step of lysis comprises the use of a detergent. The detergent may comprise NP40, DDM (n-Dodecyl-B-D-maltoside) and/or CHAP (3-[(3-cholamidopropyl)dimethylammonio]-1-propanesulfonate). A mixture of detergent may be used.

The method as defined herein may comprise detecting or measuring the level (or abundance) of non-aggregated target or aggregated target. A difference or change in the level of non-aggregated target or aggregated target as compared to a reference may, for example, indicate the presence or level of target that is bound to the ligand in the sample. For example, an increased level of non-aggregated target or a decreased level of aggregated target as compared to a reference may indicate the presence or level of target that is bound to the ligand in the sample.

The term “reference” may, for example refer to the level of non-aggregated target or aggregated target in a reference or control sample. The reference or control sample may, for example, be a sample where the ligand is not present.

The term “non-aggregated target” may refer to folded and unfolded target that is present in the sample. The method as defined herein may comprise detecting or measuring the level of “non-aggregated target”, which may include both folded and unfolded target. For example, the method may detect or measure the level of “non-aggregated target” by measuring the total amount of folded and unfolded target in a soluble fraction of a sample. In another embodiment, the method may detect or measure only the folded target. For example, the method may employ reagents (such as an antibody) that can specifically detect or measure folded target but not the unfolded or aggregated target.

In one embodiment, step c) comprises detecting or measuring the level of folded target, wherein an increased level of folded target as compared to a reference indicates the presence or level of target that is bound to the ligand in the sample

The sample may comprise living or intact cells derived from bodily fluids, blood, tissues, organoids and/or cultured cells. The sample may be a cell or tissue sample. The sample may comprise one or more cells. The cell may be a mammalian cell, a bacterial cell or a yeast cell. The sample may comprise a cell expressing a recombinant target. The recombinant target may be fused to a tag for measurement or detection of the target.

In one embodiment, the sample is one that has been obtained from a subject.

As used herein, the term “subject” includes any human or non-human animal. In one embodiment, the subject is a human. The term “non-human animal” includes all vertebrates, e.g., mammals and non-mammals, such as non-human primates, sheep, dog, cow, chickens, amphibians, reptiles, etc.

The method may comprise contacting a sample with a cell-permeable denaturant to promote intracellular unfolding of target in the sample. The term “contacting” may refer to incubating the sample with a cell-permeable denaturant for a sufficient time to unfold the target intracellularly in the sample.

The cell-permeable denaturant as referred to herein may, for example, be urea or a derivative thereof (such as thiourea or methylurea). The cell-permeable denaturant may be able to permeate intact or live cells to promote intracellular unfolding of proteins. The cell-permeable denaturant may be able to promote unfolding of intracellular or extracellular targets in or on intact or live cells.

The target may refer to any molecule that can be detected or measured using a method as defined herein. The target may be an intracellular target. In one embodiment, the target is a protein. The protein may be an intracellular protein. In another embodiment, the protein is an extracellular or membrane protein. The target may be one that is bound or associated with a nucleic acid. The target may be modified in any way, such as through post-translational modifications (e.g. phosphorylation) or by site-directed mutagenesis. The target may be a fusion protein.

The terms “protein” and “polypeptide” are used interchangeably and refer to any polymer of amino acids (dipeptide or greater) linked through peptide bonds or modified peptide bonds. Polypeptides of less than about 10-20 amino acid residues are commonly referred to as “peptides.” The polypeptides of the invention may comprise non-peptidic components, such as carbohydrate groups. Carbohydrates and other non-peptidic substituents may be added to a polypeptide by the cell in which the polypeptide is produced, and will vary with the type of cell. Polypeptides are defined herein, in terms of their amino acid backbone structures; substituents such as carbohydrate groups are generally not specified, but may be present nonetheless.

The term “polynucleotide” or “nucleic acid” are used interchangeably herein to refer to a polymer of nucleotides, which can be mRNA, RNA, CRNA, cDNA or DNA. The term typically refers to polymeric form of nucleotides of at least 10 bases in length, either ribonucleotides or deoxynucleotides or a modified form of either type of nucleotide. The term includes single and double stranded forms of DNA.

In one embodiment, the target is a recombinant protein. By “recombinant protein” is meant a protein that is made using recombinant techniques, i.e. by expression of a recombinant polynucleotide. The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

The term “ligand” as used herein refers to a molecule that can bind another molecule and includes, but is not limited to small molecules, peptides, proteins, RNA, DNA, lipids and carbohydrates. In one embodiment, the target is intracellularly bound to the ligand.

In one embodiment, the method comprises removing aggregated and/or unfolded target prior to step c). The method may involve separating the insoluble fraction from the soluble fraction. This may involve the use of microfiltration, centrifugation, affinity resins and/or microbeads. In one embodiment, centrifugation is used to pellet down insoluble suspended particles comprising aggregated target onto the bottom of vial together with cell debris. In another embodiment, affinity resins and/or microbeads may be used to remove aggregated target as well as soluble unfolded target.

In one embodiment, the method comprises removing aggregated and/or unfolded target under denaturing condition prior to step c). This enhances the removal of aggregated and/or unfolded target from the soluble fraction. In one embodiment, this enhances the removal of aggregated and/or unfolded target with micro/nano beads.

The method as defined herein may detect the presence or absence of target that is bound to the ligand in the sample. The method as defined herein may also inform of the level of target that is bound to the ligand in the sample. For example, the method may inform of the percentage of target (e.g. 10%, 20%, 30%, 40%, 50%, 60%, 70%, 80%, 90% or 100%) that is bound to the ligand in the sample.

In one embodiment, the method further comprises detecting or measuring binding of the ligand to the target at different concentrations of denaturant. The concentration of the denaturant may be any concentration that is able to induce unfolding of a target. For example, the concentration can be 0.5M, 1M, 1.5M, 2M, 2.5M, 3M, 3.5M, 4M, 4.5M, 6M, 6.5M, 7M, 7.5M, 8M, 8.5M, 9M, 9.5M, 10M, 10.5M, 11M, 11.5M, 12M, 12.5M or more.

In one embodiment, the method is performed at physiological temperature of an animal. For example, the method may be performed at around the body temperature (i.e. 37° C.) of a human.

In one embodiment, the target is coupled to a label. For example, the target may be expressed as a fusion protein with a tag. The tag may be a HIBIT tag, which is a small 11 amino acid peptide that binds with high affinity to a larger LgBiT subunit. The bound complex has luciferase activity and can be used for detection or measurement of the target.

The target may be detected by mass spectrometry (for identifying an unknown target) or by a recognition molecule (such as an antibody or aptamer). The recognition molecule may be any molecule that can recognise or bind to a target. The target may also be detected by any other bioanalytical techniques that are well known in the art. For example, the target can be detected by fluorescent protein fingerprinting, single-molecule fluorescence resonance energy transfer (FRET)-based peptide fingerprinting, or nanopore technology.

By “antibody” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include polyclonal and monoclonal antibodies as well as their fragments (such as Fab, Fab′, F(ab′)2, Fv), single chain (scFv) and domain antibodies (including, for example, shark and camelid antibodies), and fusion proteins comprising an antibody, and any other modified configuration of the immunoglobulin molecule that comprises an antigen binding/recognition site. An antibody includes an antibody of any class, such as IgG, IgA, or IgM (or sub-class thereof), and the antibody need not be of any particular class.

The term “immunoassay” as used herein refers to an analytical method which uses the ability of an antibody or antigen-binding fragment thereof to detect a target. It is contemplated that a range of immunoassay formats be encompassed by this definition, including but not limited to direct immunoassays or indirect immunoassays (including Western Blotting), and “sandwich” immunoassays (e.g. a sandwich enzyme-linked immunosorbent assay (ELISA)).

The detection of an antibody-target complex can be performed by several methods. The target may be prepared with a label such as biotin, an enzyme, a fluorescent marker, or radioactivity, and may be detected directly using this label. Alternatively, a labeled “secondary antibody” or “reporter antibody” which recognizes the primary antibody may be added, forming a complex comprised of target-antibody-antibody. Again, appropriate reporter reagents are then added to detect the labeled antibody. Any number of additional antibodies may be added as desired. These antibodies may also be labeled with a marker, including, but not limited to an enzyme, fluorescent marker, or radioactivity. Either the target or the antibody (primary or secondary) may be immobilized on a solid support, but the labeled component cannot be immobilized because the detectable signal is precluded from being a measure of binding.

As used herein, the term “reporter reagent” is used in reference to compounds which are capable of detecting the presence of antibody bound to target. For example, a reporter reagent may be a calorimetric substance which is attached to an enzymatic substrate. Upon binding of antibody and target, the enzyme acts on its substrate and causes the production of a color. Other reporter reagents include, but are not limited to fluorogenic and radioactive compounds or molecules.

As used herein, the term “solid support” is used in reference to any solid material to which reagents such as antibodies, targets, and other compounds may be attached. For example, in the ELISA method, the wells of microtiter plates often provide solid supports. Other examples of solid supports include nitrocellulose membrane, microscope slides, coverslips, beads, particles, cell culture flasks, as well as many other items.

As used herein, the terms “label” and means for detecting the antibody-target complex refer to molecules that are either directly or indirectly involved in the production of a detectable signal to indicate the presence of a complex. Any label or indicating means can be linked to or incorporated in an expressed protein, peptide, or antibody molecule that is part of the present invention, or used separately, and those atoms or molecules can be used alone or in conjunction with additional reagents. Such labels are themselves well known in clinical diagnostic chemistry.

The labeling means can be a fluorescent labeling agent that chemically binds to antibodies or targets to form a fluorochrome (dye) that is a useful immunofluorescent tracer. Suitable fluorescent labeling agents are fluorochromes such as fluorescein isocyanate (FIC), fluorescein isothiocyante (FITC), 5-dimethylamine-1-naphthalenesulfonyl chloride (DANSC), tetramethylrhodamine isothiocyanate (TRITC), lissamine, rhodamine 8200 sulphonyl chloride (RB 200 SC) and the like.

In preferred embodiments, the indicating group is an enzyme, such as horseradish peroxidase (HRP), glucose oxidase, or the like. In such cases where the principle indicating group is an enzyme such as HRP or glucose oxidase, additional reagents are required to indicate that a receptor-ligand complex (immunoreactant) has formed. Such additional reagents for HRP include hydrogen peroxide and an oxidation dye precursor such as diaminobenzidine. An additional reagent useful with glucose oxidase is 2,2,-azino-di-(3-ethyl-benzothiazoline-G-sulfonic acid) (ABTS).

Radioactive elements are also useful labeling agents and are used illustratively herein. An exemplary radiolabeling agent is a radioactive element that produces gamma ray emissions. Elements which themselves emit gamma rays, such as ¹²⁴I, ¹²⁵I, ¹²⁸I, ¹³²I and ⁵¹Cr represent one class of gamma ray emission-producing radioactive element indicating groups. Another group of useful labeling means are those elements such as ¹¹C, ¹⁸F, ¹⁵O and ¹³N which themselves emit positrons. Also useful is a beta emitter, such as ¹¹¹indium or ³H.

The linking of labels, i.e. labeling of peptides and proteins is well known in the art. For instance, monoclonal antibodies produced by a hybridoma can be labeled by metabolic incorporation of radioisotope-containing amino acids provided as a component in the culture medium. The techniques of protein conjugation or coupling through activated functional groups are particularly applicable.

Provided herein is a cell comprising a recombinant nucleic acid encoding a target fused to a tag. Provided herein is a cell comprising a target fused to a tag.

In one embodiment, the method may be used to identify an endogenous protein target that binds to a ligand (such as a bioactive compound). The method may be used for target identification and/or validation.

Disclosed herein is a method of identifying a candidate ligand that is capable of binding to a target, the method comprising: a) contacting a sample with the candidate ligand; b) contacting the sample with a cell-permeable denaturant to promote intracellular unfolding of the target; c) lysing the sample; and d) detecting or measuring the level of the non-aggregated target or aggregated target, wherein a difference in level of non-aggregated target or aggregated target as compared to a reference indicates that the candidate ligand is capable of binding to the target.

The method may be used for drug screening. For example, a cell may be screened with a drug library in a high-throughput manner to identify a candidate ligand that is capable of binding to the target.

Disclosed herein is a method of predicting the efficacy of a drug in a subject, the method comprising a) obtaining a sample from a subject who has been treated with the drug; b) contacting the sample with a cell-permeable denaturant to promote intracellular unfolding of the target; c) lysing the sample; d) detecting or measuring the level of non-aggregated target or aggregated target, wherein a difference in the level of non-aggregated target or aggregated target as compared to a reference indicates binding of the drug to the target, therefore predicting efficacy of the drug in the subject.

The method may be used to determine whether the drug reaches the target in a cellular or tissue sample that has been obtained from a patient.

The subject may be a healthy subject or a subject suffering from a condition or disease.

In one embodiment, the sample is a patient-derived cell (e.g. a patient-derived cancer cell) or a mouse xenograft.

In one embodiment, the condition or disease is a tumor or a cancer. The condition or disease may also be an infectious disease, an autoimmune disease, an inflammatory disease, or an immunodeficiency.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, blood cancer (e.g. leukemia or lymphoma), breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer.

An “infectious disease” refers to a disease that can be transmitted from person to person or from organism to organism, and is caused by a microbial agent (e.g., common cold). Infectious diseases are known in the art and include, for example, hepatitis, sexually transmitted diseases (e.g., Chlamydia, gonorrhea), tuberculosis, HIV/AIDS, diphtheria, hepatitis B, hepatitis C, cholera, influenza or a coronavirus infectious (such as by SARS-CoV-2).

An “autoimmune disease” refers to a disease in which the body produces an immunogenic (i.e., immune system) response to some constituent of its own tissue. In other words the immune system loses its ability to recognize some tissue or system within the body as “self” and targets and attacks it as if it were foreign. Autoimmune diseases can be classified into those in which predominantly one organ is affected (e.g., hemolytic anemia and anti-immune thyroiditis), and those in which the autoimmune disease process is diffused through many tissues (e.g., systemic lupus erythematosus). For example, multiple sclerosis is thought to be caused by T cells attacking the sheaths that surround the nerve fibers of the brain and spinal cord. This results in loss of coordination, weakness, and blurred vision. Autoimmune diseases are known in the art and include, for instance, Hashimoto's thyroiditis, Grave's disease, lupus, multiple sclerosis, rheumatic arthritis, hemolytic anemia, anti-immune thyroiditis, systemic lupus erythematosus, celiac disease, Crohn's disease, colitis, diabetes, scleroderma, psoriasis, and the like.

As used herein, the term “inflammatory disease” refers to either an acute or chronic inflammatory condition, which can result from infections or non-infectious causes. Various infectious causes include meningitis, encephalitis, uveitis, colitis, tuberculosis, dermatitis, and adult respiratory distress syndrome. Non-infectious causes include trauma (burns, cuts, contusions, crush injuries), autoimmune diseases, and organ rejection episodes.

An “immunodeficiency” means the state of a patient whose immune system has been compromised by disease or by administration of chemicals. This condition makes the system deficient in the number and type of blood cells needed to defend against a foreign substance. Immunodeficiency conditions or diseases are known in the art and include, for example, AIDS (acquired immunodeficiency syndrome), SCID (severe combined immunodeficiency disease), selective IgA deficiency, common variable immunodeficiency, X-linked agammaglobulinemia, chronic granulomatous disease, hyper-IgM syndrome, and diabetes.

The methods as defined herein may further comprise treating the subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The method as defined herein may also be useful in predicting the likelihood of a subject responding to a drug therapy. The method may comprise: a) obtaining a sample from a subject who has been treated with the drug; b) contacting the sample with a cell-permeable denaturant; c) lysing the sample; and d) detecting or measuring the level of non-aggregated target or aggregated target, wherein a difference in the level of non-aggregated target or aggregated target as compared to a reference predicts the likelihood of the subject responding to the drug.

Disclose herein is a method of identifying a target that is bound to a drug in a subject, the method comprising: a) obtaining a sample from a subject who has been treated with the drug; b) contacting the sample with a cell-permeable denaturant to promote intracellular unfolding of the target; c) lysing the sample; and d) detecting or measuring the level of non-aggregated target or aggregated target, wherein a difference in the level of non-aggregated target or aggregated target as compared to a reference indicates binding of the drug to the target.

The method may be used to determine whether the drug binds to a target in a cellular or tissue sample that has been obtained from a patient. The method may be used to determine whether the drug binds to a target in an intact or live cell.

In one embodiment, there is provided a method of identifying a target that is bound to a drug or ligand in a subject, the method comprising: a) obtaining a sample from a subject who has been treated with the drug; b) contacting the sample with a cell-permeable denaturant; c) lysing the sample; and d) detecting or measuring the level of non-aggregated target or aggregated target, wherein a difference in the level of non-aggregated target or aggregated target as compared to a reference indicates binding of the drug to the target.

In one embodiment, there is provided a method of identifying a target that is bound to a drug or ligand in a subject, the method comprising: a) obtaining a sample from a subject who has been treated with the drug; b) contacting the sample with a cell-permeable denaturant; c) lysing the sample; and d) detecting or measuring the target bound to a drug or ligand using mass spectrometry.

The method may be used to determine whether the drug binds to a target in an intact or live cell. The target may be an intracellular or extracellular target. The cell-permeable denaturant may promote unfolding of the intracellular or extracellular target that is present on the live or intact cell.

A microfluidic chip can be used to perform a method as defined herein. The microfluidic chip may comprise one or more microfluidic channels (e.g. 2, 3, 4, 5, 6, 7, 8, 9, 10 or more microfluidic channels). The use of microfluidics in the methods as described herein significantly reduces the amount of sample needed for detection.

Disclosed herein is a kit for performing any of the methods as defined herein. The kit may further comprise buffers, instruction manual, and the like. The kit may provide a microfluidic chip as defined herein for performing a method disclosed herein.

As used herein, “and/or” refers to and encompasses any and all possible combinations of one or more of the associated listed items, as well as the lack of combinations when interpreted in the alternative (or).

As used in this application, the singular form “a,” “an,” and “the” include plural references unless the context clearly dictates otherwise. For example, the term “an agent” includes a plurality of agents, including mixtures thereof.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

### Examples

### Methodology

### Common Steps for UCEP-ID and UCEP-Engage

### General UCEP Workflow

Living/intact cells derived from materials like body fluids, blood, tissue, organoids and cultured cells are technically compatible with the approach as described herein. However, culturing and collection of cells may need to be modified accordingly to minimize biological response, protein unfolding and protein aggregation. After treatment with bioactive compounds or vehicle for specific duration, cells were pelleted down, washed, resuspended in D-PBS or isotonic buffer containing urea or other cell-permeable chemical denaturant. In a molarity-response (MR) experiment, cells were incubated with a single concentration of drug or bioactive compound for a specific time that is typically not less than 5 minutes but tested with different concentration of urea. In current implementation, urea from 0M to 8M is used. In a dose-response (DR) experiments, cells were incubated with different concentration of drug or bioactive compound that is typically not less than 5 minutes before tested with a single concentration of chemical denaturant. Shorter drug treatment time usually detect less hits but are enriched in primary targets of drug while long treatment time allows accumulation of biological metabolized compounds and could triggers downstream events in cells.

Human cells are typically incubated with bioactive compounds and urea at 37° C. to better captured physiological state of proteins in vivo. After short incubation with urea, lysis buffer of a few times the volume of cell mixture solution was then added. Dilution factor of 2× to 4× for chemical denaturant had been tested and worked effectively with the approach. Cell lysis can be facilitated by rapid freeze-thawing process which is repeated at least 2 times. Lysed cells can be subjected to additional mechanical shearing by repeatedly passing through needle with syringe if needed.

Aggregated proteins or insoluble cell debris can be removed using several approaches like microfiltration, centrifugation, and affinity resins or microbeads. In some implementations, centrifugation is used to pellet down insoluble suspended particles onto the bottom of vial together with cell debris, and supernatant is used for downstream analysis. In another implementation, affinity resins or microbeads are used to remove both protein aggregates and soluble unfolded proteins. Microbeads have been used to capture protein aggregates to facilitate proteomics sample processing where proteins are subjected to harsh conditions to maximize protein aggregate and protein extraction. Affinity resins, microbeads and similar materials are used to separate native proteins from denatured proteins particularly small protein aggregates and soluble unfolded proteins that are not well-removed by filtration and centrifugation. Nevertheless, the use of microbeads and similar materials also has the benefit of facilitating proteomic sample preparation and enable automation.

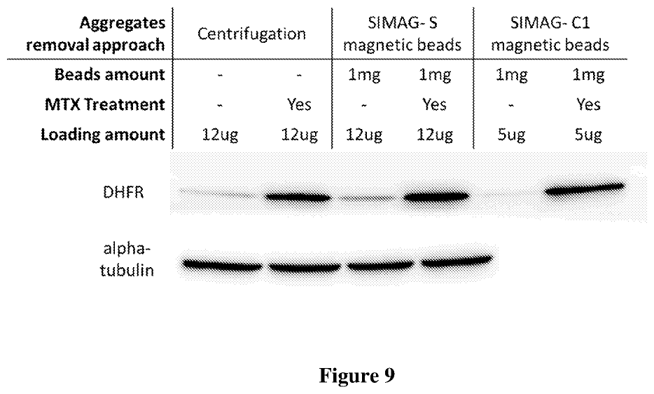

In one of the protocol development experiments using magnetic microbeads, the results showed that magnetic microbeads was remarkably efficient in separation of protein aggregates from soluble proteins fraction (FIG. 9). Protocol for aggregate separation using magnetic microbeads is start by mixing the freeze-thaw lysed cells with 1 mg of PBS pre-washed magnetic microbeads. It is subsequently incubated for 10 minutes on a rotator at room temperature. After incubation, beads-cells mixture is sitting in a strong magnetic rack for 1 minute to separate magnetic beads from the sample. Clear supernatant can be collected and snap-frozen for down-stream analysis.

### Specific Steps for UCEP-ID

### Quantitative LC-MS/MS Analysis

TCEP was added to supernatant obtained from UCEP general workflow, followed by addition of chloroacetamide. Next, “Binding” buffer (90% Methanol, 10% TEAB buffer) was added, followed by addition of phosphoric acid before samples were loaded onto “S-trap” column (Protific). After centrifugation of “S-trap” column, “washing” buffer was loaded into column to remove salts, detergents and other small impurities. Digestion buffer containing trypsin/LyC (Promega) mix was then loaded onto column. Digested peptides were eluted out from column with elution buffer after digestion. Eluted peptides were dried with speed-vac concentrator, resolubilized in TEAB buffer and labelled with Isobaric tag TMT reagents (Thermofisher).

Labelled peptides were desalted and resolubilized in solution containing 5% ammonia and 2% acetonitrile solution prior to fractionation by high pH reverse-phase chromatography in step-gradient elution mode with buffer A (10 mM ammonium formate) and Buffer B (90% ACN, 10% ammonium formate). All fractions were dried using speed-vac concentrator. Dried fractionated peptides were acidified with formic acid before loading into mass spectrometer for analysis. Acquired MS spectrums were matched against peptides using search engines like Mascot and Sequest with search parameters that include fixed modifications of peptide with carbamidomethyl and TMT-tagged, and dynamic modifications of N-terminal acetylation, methionine oxidation, and deamidation. Proteins bound by chemical have differentiated thermodynamic stability and will exhibit different intensity/abundance in treated samples compared to untreated samples, and the data generated is analyzed accordingly to identify proteins binding to chemical of interest. In DR experiment, a dose-response curve with sigmodal shape was plotted based on the measured band intensities. EC50 is calculated and defined as dose required to reach half of the maximal intensity in the dose-response curve. EC50 derived here may strongly correlate to its actual drug binding affinity in vivo.

### Specific Steps for UCEP—ENGAGE

### Western Blot/Dot—Blot/ELISA

Proteins in supernatant were denatured and reduced in sample buffer containing SDS and TCEP before gel electrophoresis analysis. Proteins in the gel were transferred onto a nitrocellulose membrane using semi-dry transfer system. After transfer, membrane was blocked with 5% milk. Membrane was subsequently probed with primary antibody before HRP-conjugated secondary antibody was added to probe the primary antibody.

Sandwich-ELISA based UCEP can also be developed for ease of handling if at least 2 antibodies that recognized different epitopes of the same protein target are available in the market. Primary antibody which coating on the ELISA plate functions of capturing the soluble target protein. ELISA plate is washed for 3 times with PBS-T to remove other uncaptured proteins. Another enzyme conjugated primary antibody is added to probe the captured protein and produce chemiluminescence signal after substrate added. ELISA-based UCEP can be further developed for automation using magnetic beads. Proteins bound by chemical will exhibit different intensity/abundance in treated samples compared to untreated samples, and data generated is then analyzed accordingly. In DR experiment, a dose-response curve with sigmodal shape was plotted based on the measured band intensities. EC50 is calculated and defined as dose required to reach half of the maximal intensity in the dose-response curve. EC50 derived here may strongly correlate to its actual drug binding affinity in vivo.

### UCEP-Screen

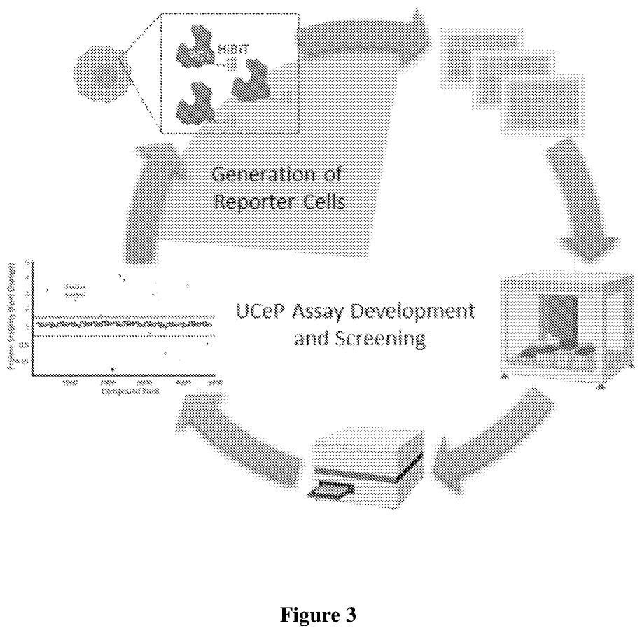

UCEP can be adapted for high-throughput screening of small molecules binding specific protein target of interest. UCEP-Screen includes steps such as generation of reporter cells, UCEP assay optimization, and screening (FIG. 3).

### Generation of Reporter Cells

In screens, cell-based assays are preferable to recombinant protein-based assays owing to a more physiologically relevant environment. Engineered reporter cells provide fast and straightforward screens. Different methods can be used to generate reporter cells. In one implementation, Flp-In T-Rex system and CRISPR are used.

In the Flp-In T-Rex system, modified pENTR1A plasmids, entry vectors, are used to clone protein of interest. pENTR1A plasmids are modified to include thirty-three nucleotide sequences of HiBiT for tagging protein of interest at either N- or C terminus. A HiBiT-tagged protein of interest is transferred to one of gateway destination vectors, pFRT/TO/DEST or pEF5/FRT/V5-DEST, via gateway LR reaction. Flp-In T-Rex HEK293 cells are transfected with the final destination vector that include HiBiT-tagged gene of interest and pOG44 vector expressing Flp recombinase. Cells are selected with Hygromycin and Blasticidin to remove untransformed cells. In the CRISPR system, the ribonucleoprotein complex including Cas9, crRNA, tracrRNA is formed in vitro. Cells are electroporated to deliver the Cas9 ribonucleoprotein complex along with a single-stranded donor oligonucleotide. Next day, cells are sorted as a single cell into a transparent 96-well tissue culture microplate and incubated until they became confluent.

### Assay Development for UCEP-Screen

UCEP conditions such as denaturant concentration and dilution factor should be optimized for each target before performing any HTS. Here, Flp-In T-Rex cells were used for assay development. Cells were seeded into the 96-well clear bottom white microplate in growth medium and the expression of HiBiT-tagged protein was induced with tetracycline. Cells are treated with chemical of interest for specific duration, before incubation in PBS containing different concentrations of urea. Urea was then diluted with PBS. HiBiT lytic detection buffer containing LgBiT and substrate was then added. Bioluminescence was then measured. Proteins bound by chemical will exhibit different intensity/abundance in treated samples compared to untreated samples, and data generated is then analyzed accordingly.

### Preliminary Results:

### UCEP—ENGAGE

### MR Experiment

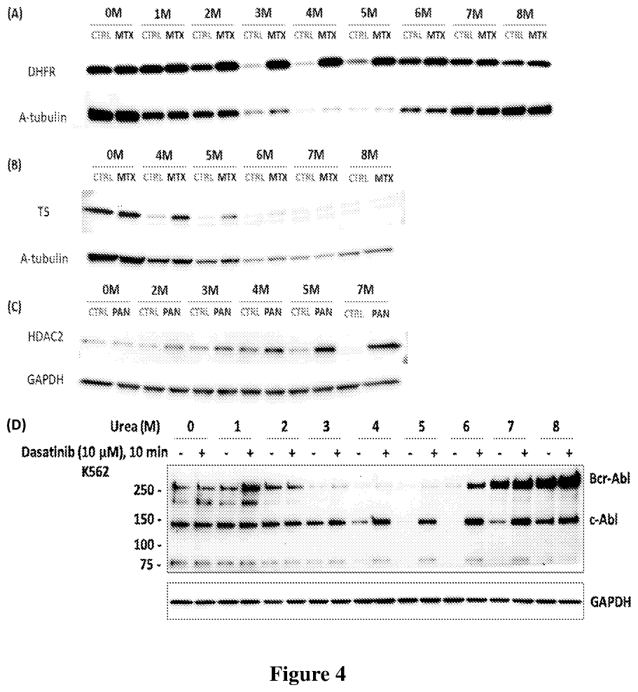

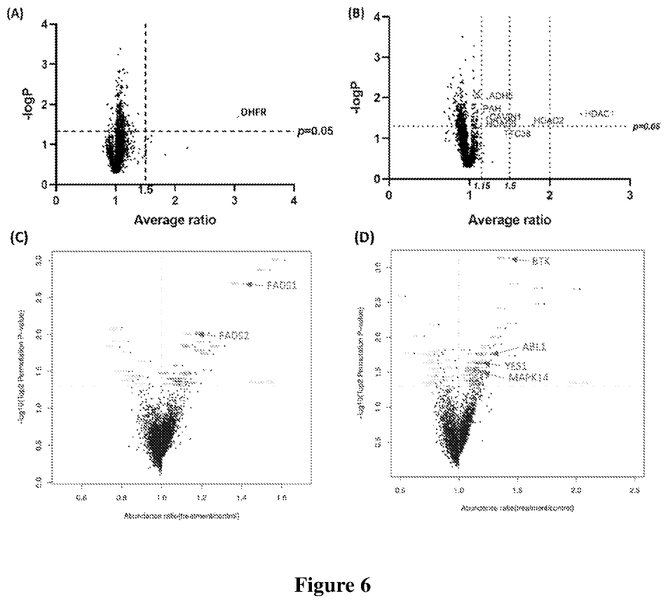

Known targets of methotrexate (MTX), dihydrofolate reductase (DHFR) and thymidylate synthase (TS) were verified and assessed by UCEP-ENGAGE. Western blot data showed that both proteins were significantly stabilized by MTX while their loading controls remained unchanged (FIGS. 4A & 4B). However, longer MTX incubation of 90 minutes was required for TS stabilization to be detected. UCEP-ENGAGE assay also verified the binding of panobinostat (PAN) to HDAC2 (FIG. 4C). The kinase inhibitor, dasatinib is developed to inhibit BCR-ABL oncogenic fusion proteins by targeting its ABL domain. Such fusion protein was present in myelogenous leukemia cell line K562. From the UCEP-ID results (FIG. 6d), ABL was detected but the unique peptide of BCR-ABL was hard to be detected by MS due to the variability of its fusion region. Therefore, UCEP-ENGAGE is useful to detect BCR-ABL based on its larger size than its normal counterparts BCR or ABL when separated by SDS-PAGE. The results (FIG. 4D) showed that dasatinib treatment increased abundance of soluble target proteins ABL as well as BCR-ABL (around 250 kDa) at urea concentrations from 3M to 6M. This suggested that UCEP can capture drug-target engagement for native proteins but also chimeric protein. Taken together, the result showed that UCEP is able to verify intracellular binding of drug to their cognate targets using detection method like western blot.

### Dose-Response (DR) Experiment

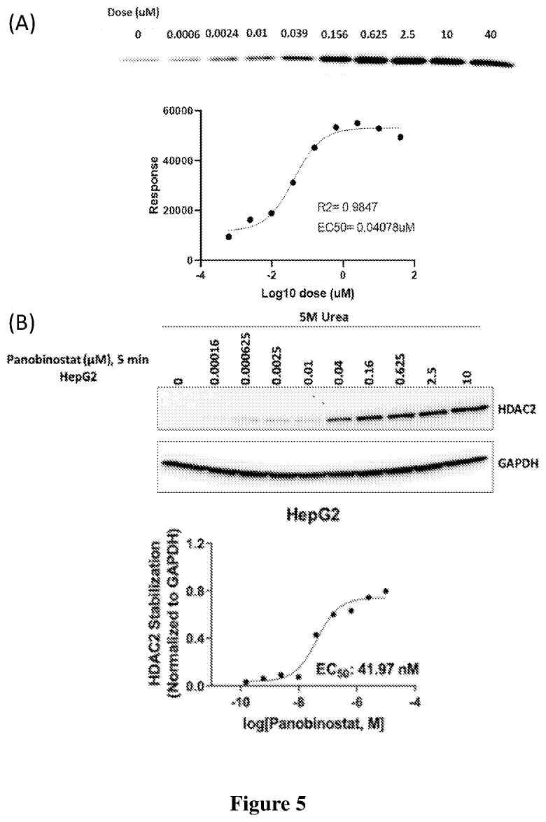

UCEP dose response experiment was performed at 4M urea condition to determine the EC50 of MTX for DHFR. An EC50 of 40 nM as calculated for MTX was determined from the dose response graph (FIG. 5A). In addition, the EC50 of another inhibitor, panobinostat, was determined for HDAC2 at 5M urea condition. Semi-quantification of its immunoblotting showed EC50 of approximately 42 nM (FIG. 5B). Calculated EC50 of MTX and PAN for DHFR and HDAC2 correlate well to other published EC50 from different assay approaches.

### UCEP-ID

A few UCEP-ID MR experiments had been carried out for methotrexate (MTX), dasatinib, and panobinostat (PAN) treatment in K562 and HEPG2 cells. Maximal dose used for MTX and dasatinib were 10 μM while PAN was 5 μM in the experiments. Compounds were incubated with cultured cells for 10 minutes except PAN, which was 90 minutes. The number of measured proteins in all the experiments were equal to or greater than 6000, in which at least 3000 proteins scored for analysis.

UCEP-ID assays identified DHFR as the binding target for MTX (FIG. 6A) and histone deacetylase 1 (HDAC1) was top-ranked target for PAN (FIG. 6A). In addition, UCEP-ID identified many other proteins as potential binding targets (off-targets) for PAN (FIG. 6b) that were also detected by 2D-TPP. This result suggests that UCEP is capable of detecting primary binding target as well as off-targets. However, two membrane proteins targets, FADS1 and FADS2 were identified in the 2D-TPP study but not by UCEP-ID. To improve the solubility of hydrophobic membrane proteins, the experiment was repeated with addition of NP-40 (final concentration was 0.4%) into the dilution buffer during cell lysis process. The results of this repeated experiment showed that protein coverage of FADS1 and FADS2 were increased and their stabilization were significantly detected (FIG. 6C).

To assess the reliability of UCEP-ID for target deconvolution of kinase inhibitors, UCEP-ID was also performed for dasatinib in K562 living cells. Dasatinib is used to treat myelogenous leukemia through inhibition of oncogenic fusion protein BCR-ABL. Target deconvolution of dasatinib by CETSA/TPP somehow failed to detect stabilization of its direct targets ABL and BTK kinases. Intriguingly, UCEP-ID successfully identified ABL kinase and BTK kinase as targets of dasatinib with highly significant p-values. Apart from that, the assay also detected other targets like YES1 and MAPK14 kinases that were previously identified by TPP method (FIG. 6D). Several studies showed that some kinases underwent conformational changes when exposed to temperature beyond 37° C. that may alter the binding site of its inhibitors resulted in reduction of their binding affinity. Therefore, UCEP is presumably useful for temperature sensitive kinases as it can be and is typically performed at physiological temperature.

### UCEP-Screen

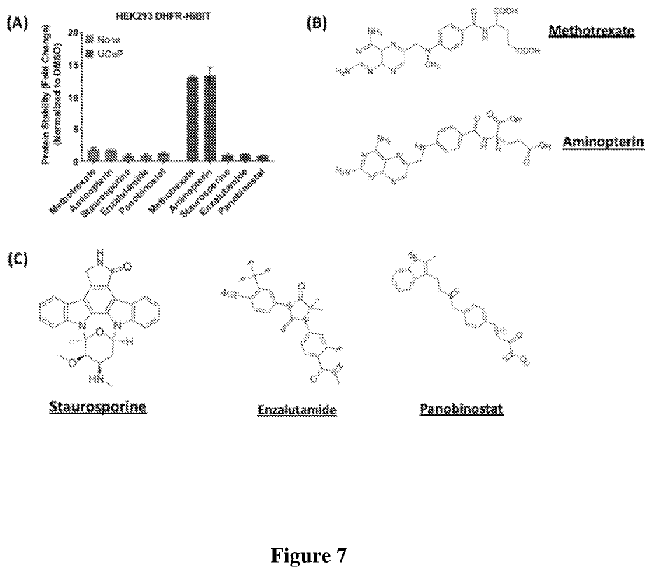

The UCEP-Screen technique was demonstrated with HiBiT-tagged dihydrofolate reductase (DHFR) to assess the selectivity of the UCEP assay with known DHFR inhibitors and non-DHFR inhibitors (FIG. 7A). DHFR inhibitors tested are Methotrexate and Aminopterin (FIG. 7B), while non-DHFR inhibitors used are Staurosporine, Enzalutamide, and Panobinostat (FIG. 7C). Methotrexate and Aminopterin dramatically increased DHFR-HiBiT protein stabilization upon UCEP while non-DHFR inhibitors did not significantly change the protein stability of DHFR-HiBiT (FIG. 7A). These findings confirm that the selective affinity of small molecules to their true protein targets can be assessed by UCEP.

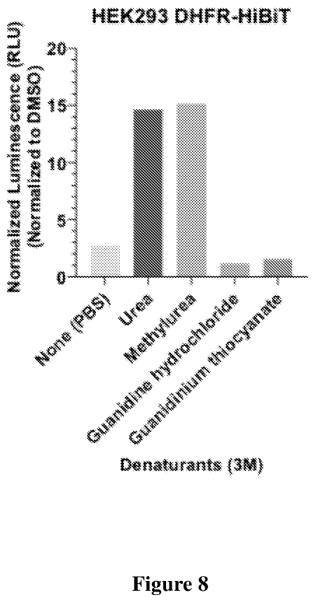

Different chemical denaturants were also evaluated during the assay development. HEK293 DHFR-HiBiT cells were treated with 20 μM Methotrexate for 10 minutes followed by incubation with different chemical denaturants such as urea, n-methylurea, guanidine hydrochloride, or guanidinium thiocyanate, at 3M concentration and two times dilution with PBS. While the assay worked well with urea and its derivative n-methylurea; other denaturants such as guanidine hydrochloride, and guanidinium thiocyanate, failed the assay (FIG. 8). This could be explained by their chemical properties. This result validates the usefulness and non-obviousness of using urea and its derivative as chemical denaturant in UCEP technology.

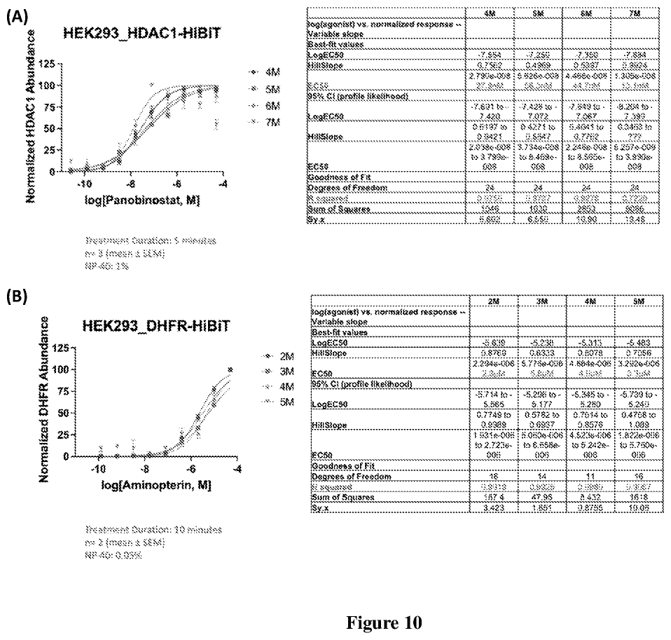

The effect of urea concentration on binding affinity in UCEP was evaluated in the HDAC1-HiBiT and DHFR-HiBiT reporter cells system (FIG. 10). They were treated with a series doses of panobinostat for 5 minutes and aminopterin for 10 minutes. The stabilization of HDAC1-HiBiT and DHFR-HiBiT by their respective inhibitor across four different effective urea concentrations was apparent. Importantly, results of both drugs demonstrate that drug binding affinities quantified with UCEP were independent of urea concentration. For example, the calculated EC50 of panobinostat was about 28 nM, 56 nM, 45 nM, and 13 nM for 4M, 5M, 6M, and 7M respectively (FIG. 10A) that are well within accepted experimental variation. Similar observation was made for aminopterin with EC50 of about 2 μM, 6 μM, 5 μM, and 3 μM for 2M, 3M, 4M, and 5M of urea respectively (FIG. 10B). This characteristic is useful for UCEP to screen a panel of drugs at a selected urea concentration with easier experimental setup and higher accuracy of EC50.

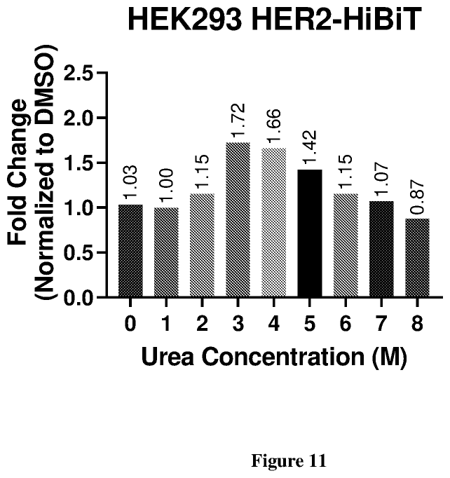

Apart from NP40 used in the UCEP-ID experiment, other non-ionic detergents like CHAPS was used to study tucatinib binding of HER2-Hibit in the reporter cell system. Tucatinib exhibited maximum stabilization of HER2 at 3M urea in this study (FIG. 11). The presence of surfactant in assay buffer or extraction buffer is important to increase the sensitivity of UCEP for membrane proteins by keeping the native form of membrane proteins soluble. However, the amount and type of surfactant used would need to be optimized to minimize solubilization of aggregated proteins and maximize the solubility of native membrane proteins.

### Results

Described herein is a novel profiling approach to identify and monitor the physical interaction of chemicals with proteins in cell lysate and in living cells and include adaptations with different downstream detection strategies for different applications. This provides novel solutions to tackle many problems faced in the pharmaceutical and biotechnology industries.

Also described herein are a series and combination of steps that permit the use of cell-permeable chemical denaturants to identify and monitor chemical-protein interactions in cell lysate and in cells. While denaturing agents like guanidinium chloride (GdmCl) has been used to study protein unfolding and protein-chemical interactions, the demonstrated that cell-permeable chemical denaturants can be used to identify and monitor intracellular chemical-protein interactions for different applications. It is also demonstrated that urea and other chemicals with similar properties, including urea derivatives like thiourea and methylurea, could be used in principle. Moreover, it is shown that naïve use of chemical denaturants cannot achieve the utilities claimed in this invention.

Pulse proteolysis using urea and protease has been applied to monitor and identify chemical-bound proteins but the approach is not applicable to identifying or monitoring chemical-protein interactions in cell as protease is not cell-permeable. Importantly, partially digested proteins will compromise the coverage and sensitivity of downstream mass spectrometry analysis commonly deployed for identifying novel binding proteins. It is demonstrated that urea can be used to identify chemical-bound proteins without protease which improve the coverage and sensitivity of downstream mass spectrometry analysis.

Recent target deconvolution approaches like CETSA have demonstrated that drug-bound proteins in cell lysate are more stable forming less insoluble aggregate upon heating. By quantifying the abundance of soluble and insoluble proteins, drug-bound protein can be determined. In contrast to heat-based assay like CETSA, the invention is independent of temperature which extend applications of the invention to both heat labile and heat resistant proteins. Data from existing heat-based approaches do not correlate well to drug binding affinity as heat or increased temperature often affects association/dissociation rate of chemical-protein interactions and decrease sensitivity for weak chemical-protein interactions. The invention cleverly uses cell-permeable chemical denaturant like urea to unfold proteins to identify drug/chemical-bound proteins in the cells under physiological temperature. Unlike existing heat-based approaches, protein unfolding induced by chemical denaturant is reversible which could be exploited to extract more binding information, for identifying weak chemical-protein interactions and differentiate indirect biological consequences in cells. The invention involving a series of steps that could be performed at physiological temperature to maximize the physiological relevance of binding information obtained but could also be combined with elevated but not denaturing temperature to enhance detection signal.

The CPP method is similar to an implementation of the invention using chemical denaturant where protein aggregation is used as a readout to identify chemical-bound proteins. However, unlike the invention, CPP uses guanidinium chloride and can only be deployed on cell lysate samples which increases false positive and false negative rates due to non-native structural conformation adopted by proteins in cell lysate. The invention can be applied directly on intact cells. It involved treating intact cells with cell-permeable denaturant and combining cell lysis and dilution of chemical denaturant in a single step to induce the aggregation of unfolded proteins. In addition, it is specifically shown that naïve usage of guanidinium chloride or other chemical denaturants is not able to achieve the utilities claimed in this invention.

In one of the implementations, cells were incubated and lyzed in similar concentration of chemical denaturant (i.e. without abrupt dilution of chemical denaturant) to minimize protein aggregation. Instead, microbeads, affinity resin or similar material to induce aggregation of proteins and for separating aggregated proteins from non-denatured proteins. The use of microbeads increases throughput and facilitate automation as it circumvents high speed centrifugation steps employed in current approaches like CETSA and CPP to remove aggregated proteins. Importantly, the use of microbeads removes unfolded proteins that could not aggregate or could not aggregated substantially for removal by centrifugation, increases the coverage and sensitivity of the method as result. In the invention, cell-permeable chemical denaturant was used to first unfold protein in cell and also to keep the protein unfolded after lysis so that the unfolded proteins are accessible for binding by the microbeads and similar materials. Proteases could also be deployed to remove unfolded protein maintained by the chemical denaturant. Another implement of the method involves combining both the dilution of chemical denaturant during cell lysis and the use of microbeads to maximize sensitivity and coverage.

Existing target deconvolution technologies based on CETSA and alike deployed protein mass spectrometry (MS) for proteome-wide identification of unknown drug targets. The principle of these technologies requires lyzing cells using a predefined “native” buffer with or without mild detergents (less than 1%). “Native” buffer is usually used to extract soluble proteins in native form. However, this may limit the extractability of many proteins that are part of some macro protein complexes or with higher hydrophobicity. In typical MS-based proteomics analysis, urea at high concentration is used to increase extraction of proteins for proteomics analysis. In the invention, urea is used at the same time for the identification of drug-bound proteins as well as for maximizing the solubility or extractability of cellular proteins from living cells. As such, the proteome coverage of the approach is higher than existing methods that extract proteins using “native” buffer.

The common proteome-wide strategy for measurement of soluble fraction in the samples is using liquid chromatography mass spectrometry. Isobaric mass tag is leveraged in the approach to measure proteome abundance of multiple samples with minimal variability compared to labelled free methods. However, limited number of label channels have been restricting the number of data points in theata analysis. In order to resolve this issue, the one-pot analysis strategy is adopted and modified by combining 2 samples as 1 sample from two adjacent conditions to include more signal information from different conditions. If dose-response analysis at proteome-wide level is favored, one-pot analysis strategy/compressed format of the approach can be employed to save MS run time.

The invention can be used in targeted protein-drug binding analysis for verifying intracellular drug occupancy or drug-target engagement. In one implementation for targeted drug-binding analysis, antibody is used to detect soluble protein but other immunoassays like western blot and ELISA can be used. If antibody is unavailable in the market, mammalian cells can be engineered to insert a small peptide tag (e.g. HIBIT, FLAG, c-Myc) onto the N-/C-terminal of targeted host protein using CRISPR-CAS technology or other cloning method (like gate-cloning). Those tagged host proteins can either activate enzymes directly or be detected by anti-tag enzyme conjugated secondary antibody to produce chemiluminescence signal.

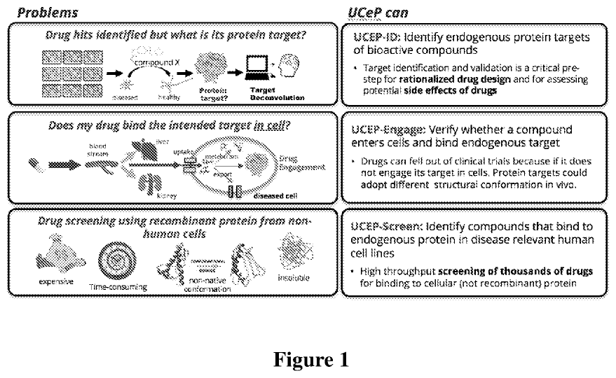

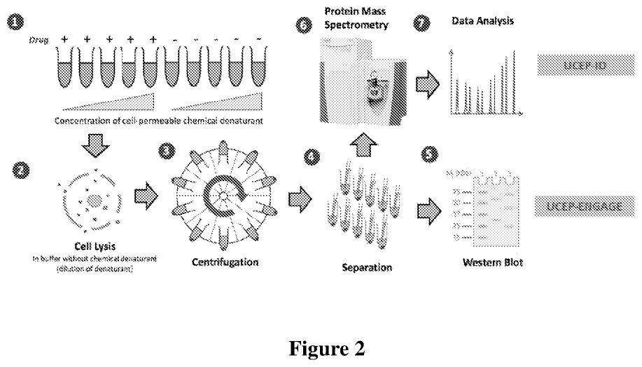

In summary, the invention named UCEP involves series and combination of steps that allow cell-permeable chemical denaturant to be used for identifying and monitoring physical interaction of chemicals with proteins in cell lysate and in living cells. It includes adaptations with different downstream detection strategies for different applications (FIG. 1). Specifically, by adapting the invention with protein mass spectrometry (termed as UCEP-ID), the invention can be used for proteome-wide analysis that allow the identification of unknown targets binding to chemical of interest (FIG. 2). UCEP-ID is useful for target deconvolution and elucidation of the mechanism-of-action of bioactive compounds in the cell. UCEP has also been adapted with targeted immunoassay, termed UCEP-ENGAGE (FIG. 2) for verifying bona fide binding of compound with a protein inside the cell. UCEP has also been adapted for high-throughput screening (HTS) for molecules binding to specific intracellular protein targets which serve to maximize physiological relevance of binding data obtained. This adaptation which includes the generation of reporter cells, the porting and optimization of UCEP assay in microplate format for cell target-based HTS is termed as UCEP-SCREEN (FIG. 3).

1. A method of detecting or measuring a target that is bound to a ligand in a sample, the method comprising:
a) contacting a sample with a cell-permeable denaturant to promote intracellular unfolding of the target in the sample;
b) lysing the sample; and
c) detecting or measuring the level of non-aggregated target or aggregated target, wherein a difference in the level of non-aggregated target or aggregated target as compared to a reference indicates the presence or level of target that is bound to the ligand in the sample.

2-15. (canceled)

16. The method of claim 1, wherein the sample comprises one or more cells.

17. The method of claim 1, wherein the sample is a cell or tissue sample.

18. The method of claim 1, wherein the cell-permeable denaturant is urea or a derivative thereof.

19. The method of claim 1, wherein the target is a protein.

20. The method of claim 1, wherein step b) comprises rapidly lysing and/or diluting the sample to promote aggregation of the target.

21. The method of claim 1, wherein the method comprises removing aggregated and/or unfolded target prior to step c).

22. The method of claim 1, wherein the method further comprises detecting or measuring binding of the ligand to the target at different concentrations of denaturant.

23. The method of claim 1, wherein the method is performed at a physiological temperature of an animal.

24. The method of claim 1, wherein the target is coupled to a label.

25. The method of claim 1, wherein the target is detected by mass spectrometry or by a recognition molecule.

26. A kit for performing the method according to claim 1.

27. A method of identifying a candidate ligand that is capable of binding to a target, the method comprising:
a) contacting a sample with the candidate ligand;
b) contacting the sample with a cell-permeable denaturant to promote intracellular unfolding of the target;
c) lysing the sample; and
d) detecting or measuring the level of the non-aggregated target or aggregated target, wherein a difference in the level of non-aggregated target or aggregated target as compared to a reference indicates that the candidate ligand is capable of binding to the target.

28. A method of predicting the efficacy of a drug in a subject, the method comprising:
a) obtaining a sample from a subject who has been treated with the drug;
b) contacting the sample with a cell-permeable denaturant to promote intracellular unfolding of a target;
c) lysing the sample; and
d) detecting or measuring the level of non-aggregated target or aggregated target, wherein a difference in the level of the non-aggregated target or aggregated target as compared to a reference indicates binding of the drug to the target, therefore predicting efficacy of the drug in the subject.

29. A method of identifying a target that is bound to a drug in a subject, the method comprising:
a) obtaining a sample from a subject who has been treated with the drug;
b) contacting the sample with a cell-permeable denaturant to promote intracellular unfolding of the target;
c) lysing the sample; and
d) detecting or measuring the level of non-aggregated target or aggregated target, wherein a difference in the level of the non-aggregated target or aggregated target as compared to a reference indicates binding of the drug to the target.
